(19) United States
(12) Patent Application Publication
(10) Pub. No.: US 2025/0195259 A1
(43) Pub. Date: Jun. 19, 2025

(54) Ostomy Device

(71) Applicant: MULTI-LOCK ApS, Haderslev (DK)

(72) Inventor: Martin Vilhelm Larsen, Vallensbæk (DK)

(21) Appl. No.: 19/070,845

(22) Filed: Mar. 5, 2025

Related U.S. Application Data
(63) Continuation of application No. PCT/DK2023/050217, filed on Sep. 7, 2023.

(30) Foreign Application Priority Data
Sep. 9, 2022 (DK) PA 2022 00830

(51) Int. Cl. A61F 5/448; A61F 5/44

(52) U.S. Cl. CPC A61F 5/448; A61F 5/4404

(57) ABSTRACT

An ostomy device for attachment (coupling) to a base plate is disclosed. The ostomy device comprises an attachment ring configured for being detachably attached to the base plate. The ostomy device further comprises a connecting element comprising one or more engagement portions configured to be detachably attached to corresponding engagement members of the attachment ring. The connecting element comprises an opening surrounded by a flange. The ostomy device comprises a sealing member configured to be detachably attached to the flange and seal the ostomy device against a collecting element extending through the opening surrounded by the flange.

### CROSS-REFERENCE TO RELATED APPLICATIONS

This application is a continuation under 35 U.S.C. 111 of International Patent Application No. PCT/DK2023/050217, filed Sep. 7, 2023, which claims the benefit of and priority to Danish Application No. PA 2022 00830, filed Sep. 9, 2022, each of which is hereby incorporated by reference in its entirety.

### FIELD OF INVENTION

The present invention relates to an ostomy device.

### BACKGROUND

In connection with certain diseases such as carcinomas of the colon or rectum, the surgical removal of the bowel (colostomy) or small intestine (ileostomy) or parts thereof is often necessary. After surgery, an artificial opening is created allowing faeces or urine, either from the intestine or the urinary tract, to pass. Discharge from the ileostomy or colostomy is collected in a stoma pouch.

Ostomy can be performed in different ways. The most common ostomies include colostomy, ileostomy and urostomy, respectively. In a colostomy operation, part of the colon is brought to the surface of the abdomen to form the stoma. There are two types of colostomy, namely end colostomy and loop colostomy. End colostomy is typically performed if parts of the colon and rectum have been removed. Loop colostomy is typically performed as a temporary measure in acute situations, and by this operation, part of the colon is lifted above skin level and held in place with a stoma rod. In an ileostomy operation, a part of the small intestine (the ileum) is brought to the surface of the abdomen to form the stoma. An ileostomy is typically created in cases where the end part of the small intestine is diseased and may be performed as either an end ileostomy or a loop ileostomy. End ileostomy is made in cases where part of the colon is removed (or needs to rest), and the end of the small intestine is brought to the surface of the abdomen to form the stoma. In a loop ileostomy, a loop of the small intestine is lifted above skin level and held in place with a stoma rod. A loop ileostomy may be temporary and performed to protect a surgical join in the bowel. By urostomy, an isolated part of the intestine is brought onto the surface of the abdomen and the other end is sewn up, and the ureters are detached from the bladder and reattached to the isolated section of the intestine. Because this section of the intestine is too small to function as a reservoir, and there is no muscle or valve to control urination, a urostomy pouch to collect the urine is needed.

It is known to apply base plates (so-called skin plates) in connection with stomas. The plates are usually fastened to the skin by an adhesive. The plates have an orifice (or it may be possible to provide an orifice) through which the stoma can be transferred. Several types of pouches to fit the base plate are known in the art.

Several ostomy devices configured to be attached to a base plate have been developed. These ostomy devices aim for reducing the risk of the skin being exposed to urine and faeces by guiding the stoma into a pouch in a smart manner. It has, however, been found that discharge from the stoma leaks and thus is present at certain parts of the ostomy device. Accordingly, these parts cannot be replaced without getting dirty hands.

U.S. Pat. No. 3,736,934 A discloses a surgical drainage appliance adapted to collect body wastes of patients who have had colostomy, ileostomy and ileum bladder operations. The appliance is positively adhered to the skin surrounding the stoma and is adapted to removably hold a disposable waste receptacle having an opening in registration with the stoma. The receptacle may be easily changed without affecting the face plate of the appliance which remains adhered to the patient without the necessity of belts and absorbent pads. It would, however, be an advantage to provide an alternative more user-friendly solution.

Thus, it is desirable to provide an ostomy device capable of limiting or even eliminating the risk of getting dirty hands when relacing parts of the ostomy device.

### BRIEF DESCRIPTION

An ostomy device according to the present disclosure is an ostomy device for attachment (coupling or gluing) to a base plate, wherein the ostomy device comprises an attachment ring configured for being detachably attached to the base plate, wherein the ostomy device further comprises a connecting element comprising one or more engagement portions configured to be detachably attached to corresponding engagement members of the attachment ring, wherein the connecting element comprises an opening surrounded by a flange, wherein the ostomy device comprises a sealing member configured to be detachably attached to the flange and seal the ostomy device against a collecting element extending through the opening surrounded by the flange, wherein the flange comprises a radially protruding protrusion configured to engage with an annular outer portion of the sealing member, wherein the annular outer portion is shaped to be detachably attached to the flange.

Hereby, it is possible to provide an ostomy device capable of limiting or even eliminating the risk of getting dirty hands when replacing parts of the ostomy device.

An ostomy device according to the present disclosure is an ostomy device for attachment (coupling or gluing) to a base plate. Accordingly, the ostomy device is applicable in a large range of solutions.

In an embodiment, the ostomy device is configured to be glued to a base plate.

In an embodiment, the attachment ring is configured to be glued to a base plate.

The ostomy device comprises an attachment ring configured for being detachably attached to the base plate. The attachment ring may be detachably attached to an annular structure of the base plate.

The ostomy device further comprises a connecting element comprising one or more engagement portions configured to be detachably attached to corresponding engagement members of the attachment ring. Hereby, the connecting element can be placed on the top of the attachment ring and be detachably attached thereto.

The connecting element comprises an opening surrounded by a flange. Accordingly, the stoma and a collecting element (e.g. a pouch) can extend through said opening.

The ostomy device comprises a sealing member configured to be detachably attached to the flange and seal the ostomy device against a collecting element extending through the opening surrounded by the flange. Hereby, the sealing member can seal the ostomy device against a collecting element and hereby prevent critical parts of the ostomy device from getting dirty.

In an embodiment, the sealing member comprises a wall portion defining a hole configured to receive the collecting element, wherein the wall portion is made of a resilient material.

In an embodiment, the sealing member is made of a resilient material, such as rubber or silicone.

In an embodiment, the collecting element is a pouch.

In an embodiment, the wall portion is conical.

In an embodiment, the wall portion is cylindrical.

It is an advantage that the flange comprises a radially protruding protrusion configured to engage with an annular outer position of the sealing member, wherein the annular outer portion is shaped to be detachably attached to the flange.

In an embodiment, the height H₂ of the wall portion is larger than the height of the attachment ring and the connecting element stacked on top of each other and attached to one another.

By the term “attachment ring and the connecting element stacked on top of each other” is meant that the connecting element is attached on the top of the attachment ring.

In an embodiment, the height H₂ of the wall portion is larger than the height H₁ of the outer portion.

In an embodiment, the distal end of the wall portion protrudes from the outer portion with respect to the longitudinal axis of the sealing member. Hereby, the wall portion is capable of guiding the pouch in an advantageous manner.

In an embodiment, the sealing member comprises an open cap provided with an attachment structure extending along an inner wall of the open cap, wherein the open cap is provided with an open end configured and shaped to allow a stoma to extend out of the open end of the open cap. Hereby, the open cap protects the pouch and/or stoma against impact and pressure exerted from outside.

In an embodiment, the sealing member comprises a grip portion protruding radially from the outer portion. Hereby, the grip portion can be used when attaching and/or detaching the sealing member.

In an embodiment, the opening in the sealing member is surrounded by a portion provided with annular markings intended to indicate cutting lines for cutting a larger opening.

In an embodiment, the sealing member comprises a planar bottom portion that is aligned with a bottom part of the attachment ring.

In an embodiment, the connecting element comprises two opposing guiding elements extending along the periphery of a plate-shaped portion of the connecting element. Hereby, the open cap can be detachably attached (and detached) in an easy manner.

In an embodiment, a protruding structure protrudes from the central portion of each of the guiding elements.

In an embodiment, several spaced apart notches are provided in the periphery of a plate-shaped portion of the connecting element.

In an embodiment, the notches are provided in opposite ends of the connecting element.

### BRIEF DESCRIPTION OF THE DRAWINGS

The present devices will become more fully understood from the detailed description given herein below. The accompanying drawings are given by way of illustration only, and thus, they are not limitative. In the accompanying drawings:

FIG. 1A shows a perspective top view of an ostomy device according to an embodiment;

FIG. 1B shows an exploded view of the ostomy device shown in FIG. 1A;

FIG. 2A shows a cross-sectional view of an ostomy device according to an embodiment;

FIG. 2B shows another view of the ostomy device shown in FIG. 2A;

FIG. 3A shows a person attaching a sealing member 10 to an ostomy device according to an embodiment;

FIG. 3B shows the ostomy device shown in FIG. 3A in a configuration in which the sealing member 10 has been detachably attached to the ostomy device;

FIG. 4A shows a cross-sectional view of an ostomy device according to an embodiment;

FIG. 4B shows a cross-sectional view of an ostomy device according to an embodiment;

FIG. 5A shows a cross-sectional view of an ostomy device according to an embodiment;

FIG. 5B shows a cross-sectional view of an ostomy device according to an embodiment;

FIG. 6A shows a perspective view of a sealing member according to an embodiment;

FIG. 6B shows a perspective view of an ostomy device according to an embodiment;

FIG. 7A shows a perspective view of a sealing member according to an embodiment;

FIG. 7B shows a perspective view of an ostomy device according to an embodiment;

FIG. 8A shows a cross-sectional view of an ostomy device according to an embodiment;

FIG. 8B shows a cross-sectional view of an attachment ring of an ostomy device according to an embodiment arranged above a base plate; and

FIG. 8C shows the attachment ring shown in FIG. 8B in a configuration in which the attachment ring has been attached to the base plate.

### DETAILED DESCRIPTION

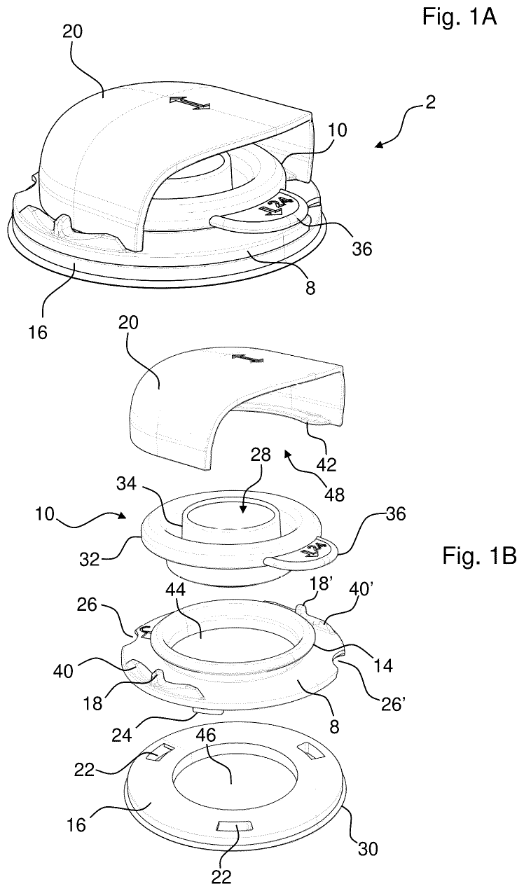

Referring now in detail to the drawings for the purpose of illustrating embodiments of the present devices, an ostomy device 2 is illustrated in FIG. 1A.

FIG. 1A illustrates a perspective top view of an ostomy device according to an embodiment, whereas FIG. 1B illustrates an exploded view of the ostomy device shown in FIG. 1A.

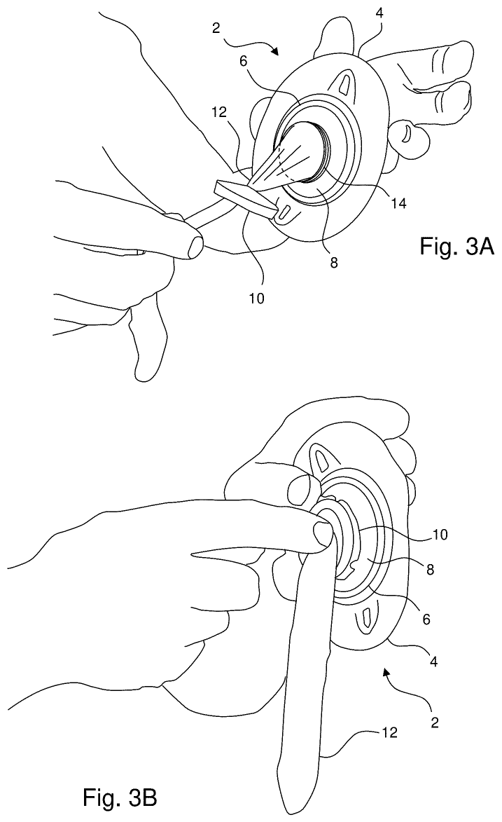

The ostomy device 2 is designed for being attached to a base plate (see FIG. 3A and FIG. 3B).

The ostomy device 2 comprises an attachment ring 16 configured to be detachably attached to the base plate 6. The ostomy device 2 comprises a connecting element 8 comprising one or more engagement portions 24 configured to be detachably attached to corresponding engagement members of the attachment ring 16 hereby detachably attaching the connecting element 8 to the attachment ring 16. The attachment ring 16 is provided with a plurality of engagement members shaped as openings 22 configured to receive and hereby be detachably attached to the engagement portions 24 of the connecting element 8.

The attachment ring 16 comprises an opening 46 shaped to allow a stoma to enter.

The connecting element 8 comprises an opening 44 surrounded by a flange 14 provided with an attachment profile.

The ostomy device 2 comprises a sealing member 10 configured to be detachably attached to the flange 14 and seal against a collecting element (see FIG. 3A and FIG. 3B) extending through the opening 44 surrounded by the flange 14.

The attachment profile of the flange 14 comprises a radial protrusion configured to engage with an outer position 32 of the sealing member 10.

The connecting element 8 comprises a plate-shaped portion provided with a centrally arranged opening 44 defined by the flange 14. Several notches 26, 26′ are provided in the periphery of the plate-shaped portion of the connecting element 8. The notches 26, 26′ are provided in opposite ends of the connecting element 8.

The connecting element 8 comprises two opposing guiding elements 40, 40′ extending along the periphery of the plate-shaped portion of the connecting element 8. A protruding structure 18, 18′ protrudes from the central portion of each of the guiding elements 40, 40′.

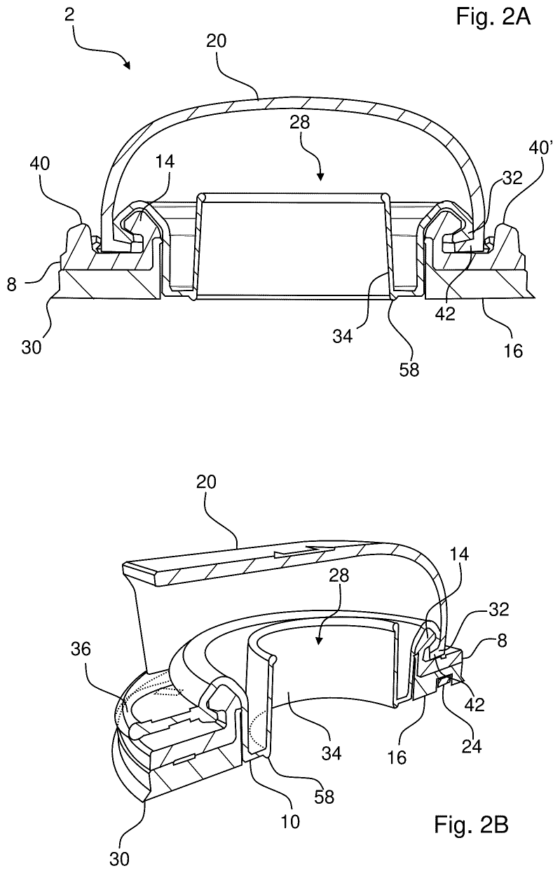

The sealing member 10 is made of a resilient material (e.g. an elastomer) and comprises a hole 28 defined by wall portion 34. A grip portion 36 protrudes from the outer position 32 of the sealing member 10. The grip portion 36 is configured to be used when attaching and detaching the sealing member 10. The outer position 32 of the sealing member 10 is configured to be detachably attached to the flange 42 in such a manner that the outer position 32 engages the radial protrusion of the flange (see FIG. 2A and FIG. 2B for a better view).

The ostomy device 2 comprises an open cap 20 provided with an attachment structure 42 extending along an inner wall of the open cap 20. The open cap 20 is provided with an open end 48 allowing the stoma to extend out of the open cap 20.

The attachment structure 42 is configured to engage with the sealing member 10 or the radial protrusion of the flange 14 (see FIG. 2A) and hereby be detachably attached to the connecting element 8.

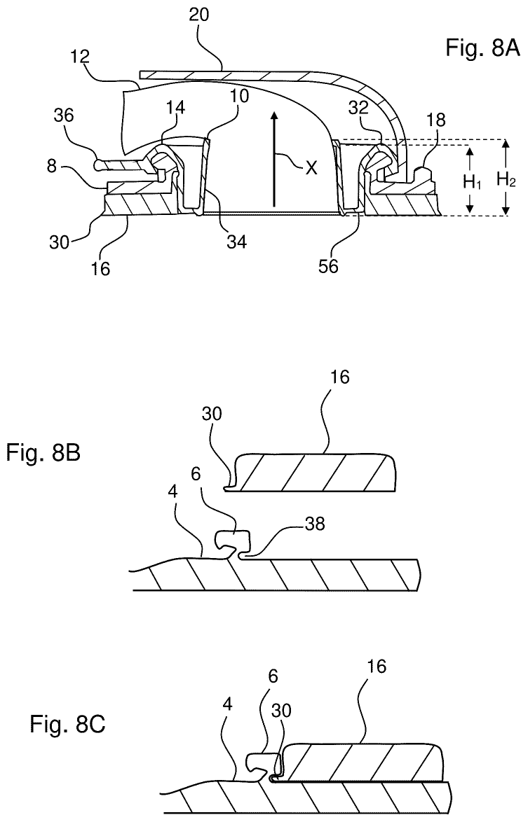

FIG. 2A illustrates a cross-sectional view of an ostomy device 2 according to an embodiment and FIG. 2B illustrates another view of the ostomy device shown in FIG. 2A. The ostomy device 2 corresponds to the one shown in FIG. 1A and FIG. 1B. The ostomy device 2 comprises an attachment ring 16 provided with an inclined ring portion 30. The inclined ring portion 30 extends along the periphery of the lower portion of the attachment ring 16. The inclined ring portion 30 is configured to be inserted to a corresponding cavity 38 as shown in FIG. 8B and FIG. 8C.

The attachment ring 16 is provided with a central opening. The attachment ring 16 is detachably attached to a connecting element 8 comprising a plate-formed annular portion that is attached to the attachment ring 16 by attachment structures corresponding to the ones shown in and explained with reference to FIG. 1A and FIG. 1B.

The connecting element 8 comprises two protruding structures protruding from the central portion of each of the guiding elements 40, 40′ of the connecting element 8. The protruding structures are arranged and configured to guide and maintain the open cap 20 in a fixed position, in which the open cap 20 is attached to the connecting element 8. It can be seen that the open cap 20 comprises an attachment structure 42 that abuts the upper surface of the annular plate-shaped portion of the connecting element 8. The attachment structure 42 abuts the outer portion 32 of the sealing member 10.

The outer portion 32 of the sealing member 10 is attached to the flange 14 of the connecting element 8. This can be done because the outer portion 32 has a concave portion arranged and configured to engage with the convex portion (protruding radially from the remaining portion) of the flange 14.

The sealing member 10 may be made of silicone or another resilient material. The sealing member 10 comprises a centrally arranged hole 28 extending through the entire height of the sealing member 10. The hole 28 is defined by a slightly conical wall portion 34 having the narrowing portion in the top. The wall portion 34 may be cylindrical.

In FIG. 2B it can be seen that the connecting element 8 comprises an engagement portion 24 that has been detachably attached to a corresponding engagement portion (opening) in the attachment ring 16.

Moreover, it can be seen that the sealing member 10 comprises a grip portion 36 protruding radially from the outer portion 32 of the sealing member 10. It can be seen that the open cap 20 comprises an open end 48. The grip portion 36 protrudes radially from the outer portion 32 of the sealing member 10 out through the open end 48 of the open cap 20.

The sealing member 10 comprises a ring-shaped extended portion 58 protruding from the bottom part of the wall portion 34. The extended portion 58 is arranged and configured to seal against the proximal portion of a stoma. This function is in particular important for persons that have a short stoma. The extended portion 58 is arranged and configured to guide the stoma into the hole 28 and ensure that the stoma is maintained in a position in which the stoma extends into the hole 28.

In an embodiment, the extended portion 58 has a height in the range 0.5-3.0 mm. In an embodiment, the extended portion 58 has a height in the range 1.0-2.0 mm. In an embodiment, the extended portion 58 has a height in the range 1.2-1.8 mm. In an embodiment, the extended portion 58 has a height in the range 1.4-1.6 mm. In an embodiment, the height of the extended portion 58 is 1.5 mm.

In an embodiment, the extended portion 58 has a width in the range 0.5-3.0 mm. In an embodiment, the extended portion 58 has a width in the range 1.0-2.0 mm. In an embodiment, the extended portion 58 has a width in the range 1.2-1.8 mm. In an embodiment, the extended portion 58 has a width in the range 1.4-1.6 mm. In an embodiment, the width of the extended portion 58 is 1.5 mm.

FIG. 3A illustrates a person attaching a sealing member 10 to an ostomy device 2 according to an embodiment. The ostomy device 2 comprises a connecting element 8 corresponding to the one shown in and explained with reference to FIG. 1A, FIG. 1B, FIG. 2A and FIG. 2B. The connecting element 8 is attached to an attachment ring (not shown) like the one shown in and explained with reference to FIG. 1A, FIG. 1B, FIG. 2A and FIG. 2B. The connecting element 8 is attached to an annular structure 6 of a base plate 4 that is intended to be attached to a patient.

It can be seen that the connecting element 8 comprises a flange 14. A collecting element (a flexible bag) 12 extends through the opening in the connecting element 8. The collecting element 12 extends through a centrally arranged opening in a sealing member 10.

FIG. 3B illustrates the ostomy device shown in FIG. 3A in a configuration, in which the sealing member 10 has been detachably attached to the ostomy device 2. The sealing member 10 has been attached to the flange 14.

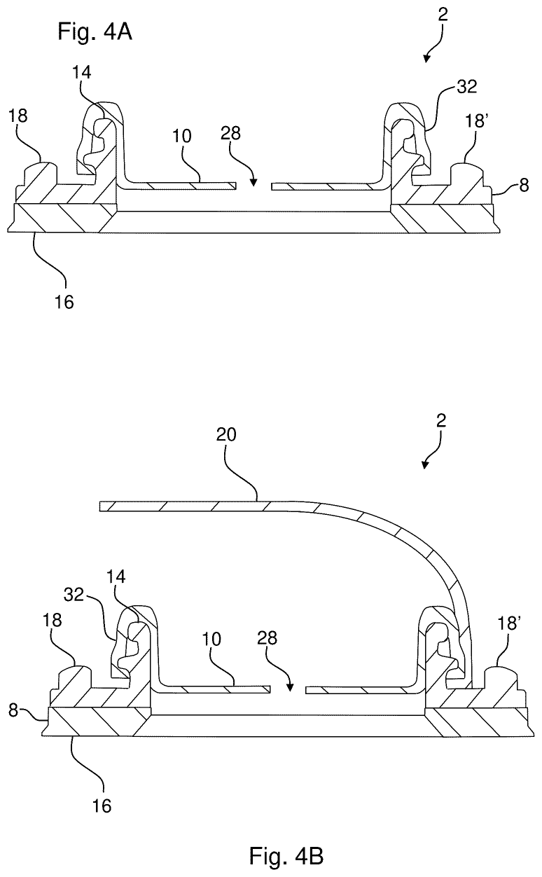

FIG. 4A illustrates a cross-sectional view of an ostomy device 2 according to an embodiment. The ostomy device 2 comprises an attachment ring 16 that is attached to a connecting element 8. Both the attachment ring 16 and the connecting element 8 comprise a centrally arranged opening through which a stoma can extend.

The connecting element 8 comprises two opposing protruding structures 18, 18′. The connecting element 8 comprises a flange 14 provided with outer connection structures adapted to engage with the outer portion 32 of the sealing structure 10. The sealing structure 10 comprises a central hole 28. This hole 28 is relatively small. The hole 28 can, however, be enlarged by cutting away a portion surrounding the hole 28 of the sealing member 10 e.g. with a knife or scissors.

FIG. 4B illustrates a cross-sectional view of the ostomy device 2 shown in FIG. 4A. The ostomy device 2 comprises, however, an open cap 20 that has been attached to the remaining portion of the ostomy device 2.

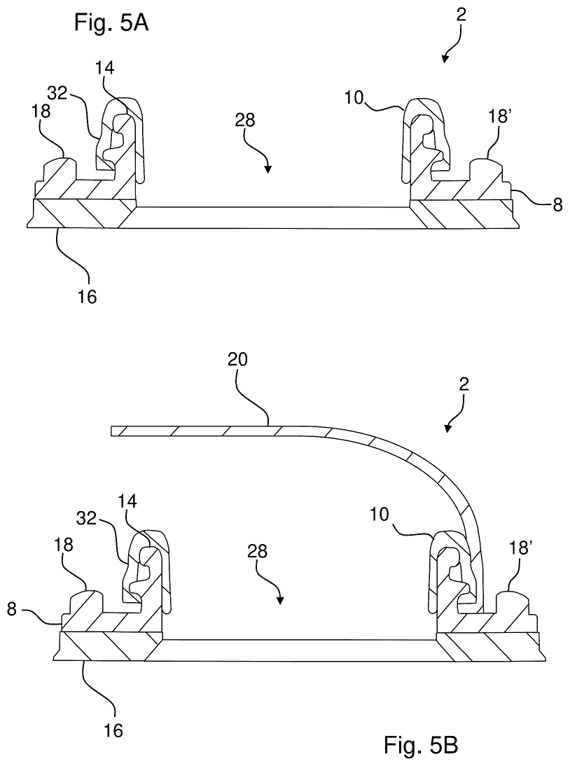

FIG. 5A illustrates a cross-sectional view of an ostomy device 2 according to an embodiment. The ostomy device 2 comprises an attachment ring 16 that is attached to a connecting element 8. The attachment ring 16 and the connecting element 8 correspond to the ones shown in and explained with reference to FIG. 4A and FIG. 4B. The ostomy device 2 comprises a sealing member that comprises an outer portion 32 that is attached to the flange 14 of the connecting element 8 as shown and explained with reference to FIG. 4A. The hole 28 of the sealing member 10, however, is much larger than in FIG. 4A.

FIG. 5B illustrates a cross-sectional view of the ostomy device 2 shown in FIG. 5A. The ostomy device 2 comprises, however, an open cap 20 that has been attached to the remaining portion of the ostomy device 2.

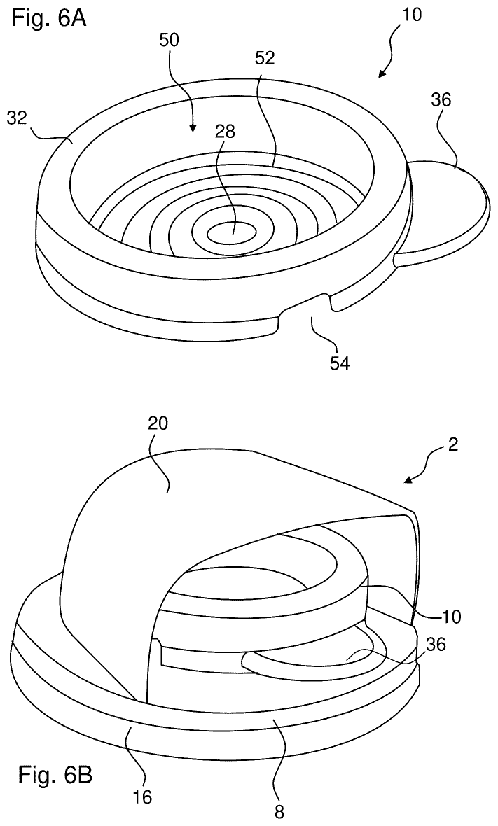

FIG. 6A illustrates a perspective view of a sealing member 10 according to an embodiment. The sealing member 10 comprises a bowl-formed portion comprising a cavity 50 provided with markings 52. The sealing member 10 comprises an outer portion 32 provided with an indentation 54. The sealing member 10 comprises a grip portion 36 extending radially from the outer portion 32.

FIG. 6B illustrates a perspective view of an ostomy device 2 according to an embodiment. The ostomy device 2 comprises a sealing member 10 corresponding to the one shown in FIG. 6A. The ostomy device 2 comprises an attachment ring 16, a connecting element 8, a sealing member 10 and an open cap 20 corresponding to the ones shown in FIG. 1A, FIG. 1B, FIG. 2A and FIG. 2B.

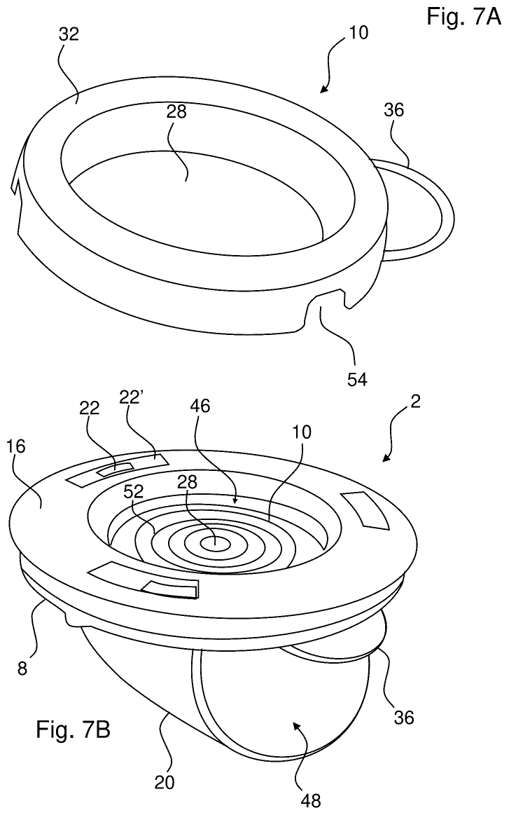

FIG. 7A illustrates a perspective view of a sealing member 10 according to an embodiment. The sealing member 10 corresponds to the one shown in FIG. 5A and FIG. 5B. The sealing member 10 comprises an outer portion 32 and a hole 28. A grip portion 36 protrudes from the outer portion 32. The outer portion 32 comprises several indentations 54.

FIG. 7B illustrates a perspective view of an ostomy device 2 according to an embodiment. The ostomy device 2 corresponds to the one shown in FIG. 6B. The ostomy device 2 comprises an attachment ring 16 comprising an opening 46 and a plurality of openings 22. A receiving structure 22′ is arranged next to each opening 22. Each receiving structure 22′ is configured to be brought into engagement with an engagement portion of the connecting element 8. The connecting element 8 is attached to the attachment ring 16.

The ostomy device 2 comprises a sealing member 10 corresponding to the one shown in FIG. 6A. The sealing member 10 comprises a hole 28 and a plurality of markings 52. The ostomy device 2 comprises an open cap 20 corresponding to the one shown in FIG. 6B. A grip portion 36 extends through the opening 48 of the open cap 20. The grip portion 36 protrudes radially from the outer portion 32 of the sealing member 10.

FIG. 8A illustrates a cross-sectional view of an ostomy device 2 according to an embodiment. The ostomy device 2 basically corresponds to the one shown in FIG. 5B. A collecting element 12 formed as a pouch extends through the hole in the sealing member 10 and through the open end of the open cap 20. The sealing member 10 has a longitudinal axis X.

It can be seen that the sealing member 10 seals against the collecting element 12. The height H₂ of the wall portion 34 of the sealing member 10 is larger than the attachment ring 16 and the connecting element 8 stacked on top of each other (the connecting element 8 attached on the top of the attachment ring 16). The height H₂ of the wall portion 34 of the sealing member 10 is larger than the height H₁ of the outer portion 32.

The sealing member 10 comprises a planar bottom portion 56 that is aligned with the bottom part of the attachment ring 16.

FIG. 8B illustrates a cross-sectional view of an attachment ring 16 of an ostomy device according to an embodiment arranged above a base plate 4. The base plate 4 comprises an annular structure 6 provided with a cavity 38 configured to receive the ring portion 30 of the attachment ring 16.

FIG. 8C illustrates the attachment ring 16 shown in FIG. 8B in a configuration in which the attachment ring 16 has been attached to the base plate 4.

### LIST OF REFERENCE NUMERALS

2 Ostomy device
4 Base plate
6 Annular structure
8 Connecting element
10 Sealing member
12 Collecting element
14 Flange
16 Attachment ring
18 Protruding structure
20 Open cap
22 Opening
22′ Receiving structure
24 Engagement portion
26, 26′ Notch
28 Hole
30 Ring portion
32 Outer portion
34 Wall portion
36 Grip portion
38 Cavity/recess
40, 40′ Guiding element
42 Attachment structure
44, 46 Opening
48 Open end
50 Cavity
52 Marking
54 Indentation
56 Bottom portion
58 Extended portion
H₁, H₂ Height
X Longitudinal axis

What is claimed is:

1. An ostomy device for attachment to a base plate, the ostomy device comprising:
an attachment ring configured for being detachably attached to the base plate;
a connecting element comprising one or more engagement portions configured to be detachably attached to corresponding engagement members of the attachment ring, wherein the connecting element comprises an opening surrounded by a flange;
a sealing member configured to be detachably attached to the flange and to seal the ostomy device against a collecting element extending through the opening surrounded by the flange;
wherein the flange comprises a radially protruding protrusion configured to engage with an annular outer portion of the sealing member, wherein the annular outer portion is shaped to be detachably attached to the flange.

2. The ostomy device according to claim 1, wherein the sealing member comprises a wall portion defining a hole configured to receive the collecting element.

3. The ostomy device according to claim 2, wherein a height (H₂) of the wall portion is larger than a height of the attachment ring and the connecting element stacked on top of each other and attached to one another.

4. The ostomy device according to claim 3, wherein the height (H₂) of the wall portion is larger than a height (H₁) of the annular outer portion.

5. The ostomy device according to claim 2, wherein the height (H₂) of the wall portion is larger than a height (H₁) of the annular outer portion.

6. The ostomy device according to claim 2, wherein a distal end of the wall portion protrudes from the annular outer portion with respect to a longitudinal axis (X) of the sealing member.

7. The ostomy device according to claim 1, wherein the sealing member comprises an open cap provided with an attachment structure extending along an inner wall of the open cap, wherein the open cap is provided with an open end configured and shaped to allow a stoma to extend out of the open end of the open cap.

8. The ostomy device according to claim 1, wherein the sealing member comprises a grip portion protruding radially from the annular outer portion.

9. The ostomy device according to claim 1, wherein the sealing member comprises a planar bottom portion that is aligned with a bottom part of the attachment ring.

10. The ostomy device according to claim 1, wherein the connecting element comprises two opposing guiding elements extending along a periphery of a plate-shaped portion of the connecting element.

11. The ostomy device according to claim 10, further comprising a protruding structure protruding from a central portion of each of the guiding elements.

12. The ostomy device according to claim 10, further comprising several spaced apart notches provided in the periphery of the plate-shaped portion of the connecting element.
